(19) United States
(12) Patent Application Publication
(10) Pub. No.: US 2019/0225114 A1
(43) Pub. Date: Jul. 25, 2019

(54) HEALTH MONITORING CAR SEAT ASSEMBLY

(71) Applicant: Kenyatta Bellamy, Davenport, FL (US)

(72) Inventor: Kenyatta Bellamy, Davenport, FL (US)

(21) Appl. No.: 15/878,138

(22) Filed: Jan. 23, 2018

(51) Int. Cl. B60N 2/00; B60N 2/26; B60R 11/02; A61B 5/00; A61B 5/0205; A61F 7/00; A61M 21/00

(52) U.S. Cl. CPC B60N 2/002; B60N 2/26; B60R 11/0235; B60R 11/0258; B60R 11/0217; B60R 2011/0012; A61B 5/02055; A61B 5/7445; A61F 7/00; A61M 21/00; A61B 5/6893

(57) ABSTRACT

A health monitoring car seat assembly for monitoring the health of a child thereon includes a child safety seat that receives a child thereon. A housing is attached to the child safety seat. A sensing unit is mounted on the housing. The sensing unit detects vital signs of a mass in the child safety seat when the mass is a child. The vital signs include body temperature and heart rate. The sensing unit also detects an ambient temperature of air adjacent to the child safety seat. A display is mounted on the child safety seat and is electrically coupled to the sensing unit. The display displays each of the ambient temperature, the temperature of the child, the heart rate of the child and the weight of the child. A power supply is mounted in the housing and electrically coupled to the sensing unit and the display.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to car seat devices and more particularly pertains to a new car seat device for monitoring the health of a child thereon.

### BRIEF SUMMARY OF THE INVENTION

An embodiment of the disclosure meets the needs presented above by generally comprising a child safety seat that has an upper surface and a lower surface. The upper surface is configured to receive a child thereon. A housing is attached to the child safety seat. A sensing unit is mounted on the housing. The sensing unit is configured to detect when a mass is positioned in the child safety seat and is further configured to detect vital signs of the mass in the child safety seat when the mass is a child. The vital signs include body temperature and heart rate. The sensing unit also detects an ambient temperature of air adjacent to the child safety seat. A display is mounted on the child safety seat and is electrically coupled to the sensing unit. The display displays each of the ambient temperature, the temperature of the child, the heart rate of the child and the weight of the child. A power supply is mounted in the housing. The power supply is electrically coupled to the sensing unit and the display.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front side view of a health monitoring car seat assembly according to an embodiment of the disclosure.

FIG. 2 is a front view of an embodiment of the disclosure.

FIG. 3 is a back view of an embodiment of the disclosure.

FIG. 4 is a cross-sectional view of an embodiment of the disclosure taken along line 4-4 of FIG. 2.

FIG. 5 is a block diagram view of an embodiment of the disclosure.

### DETAILED DESCRIPTION OF THE INVENTION

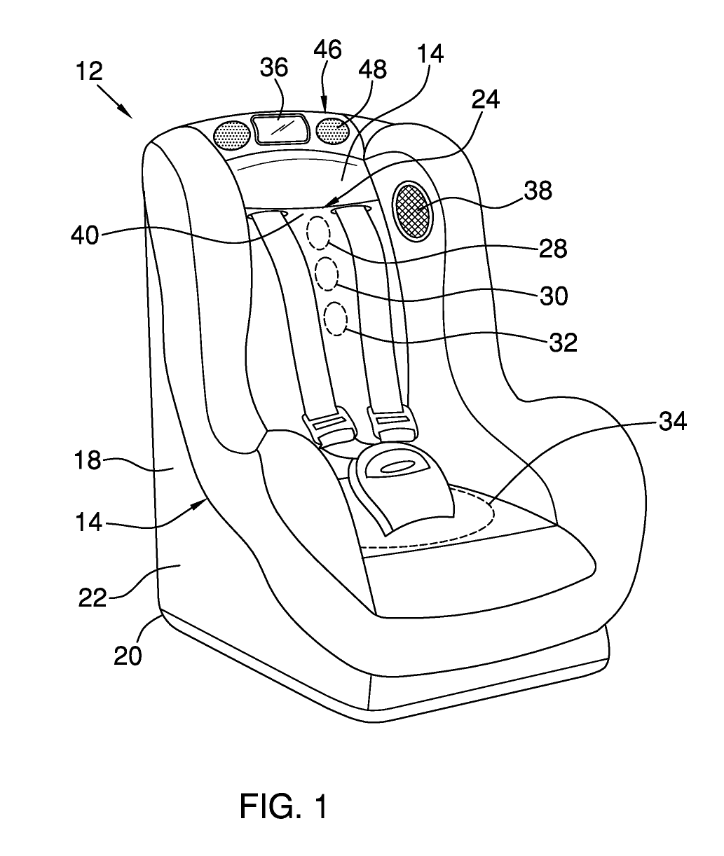

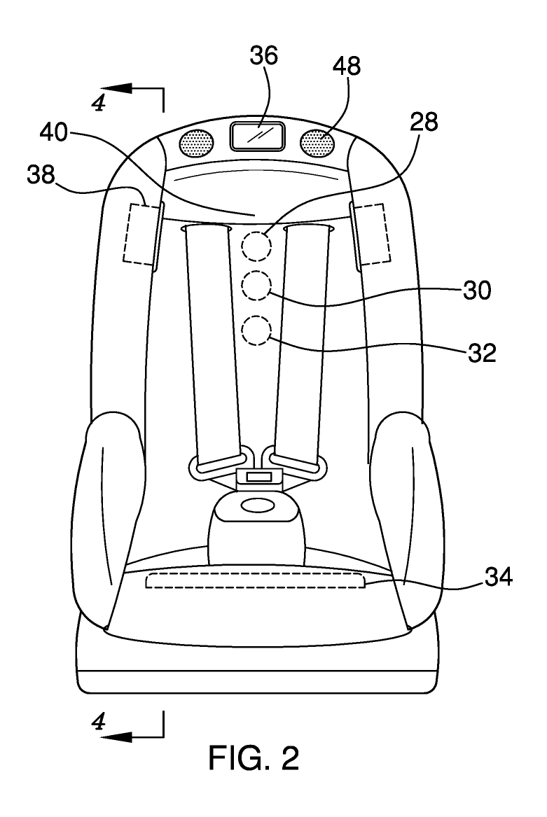

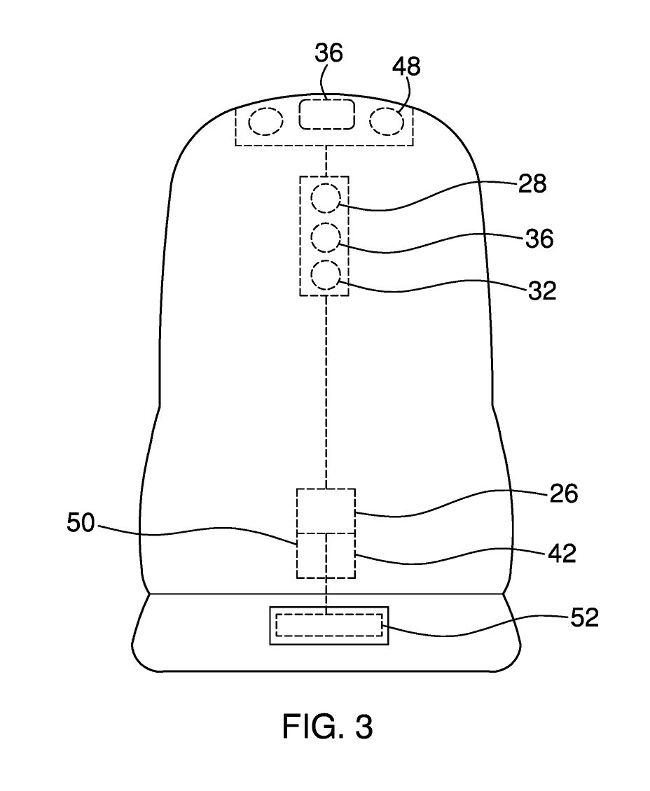

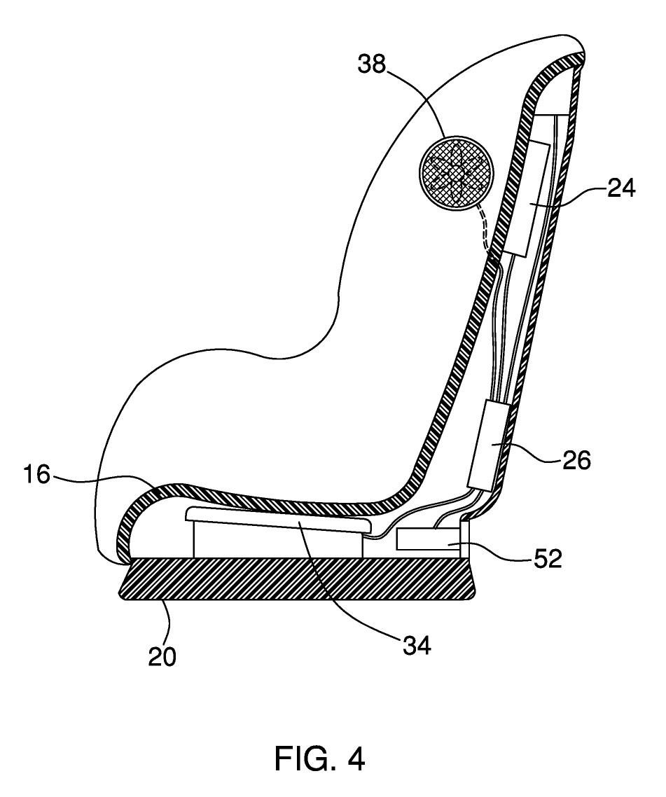

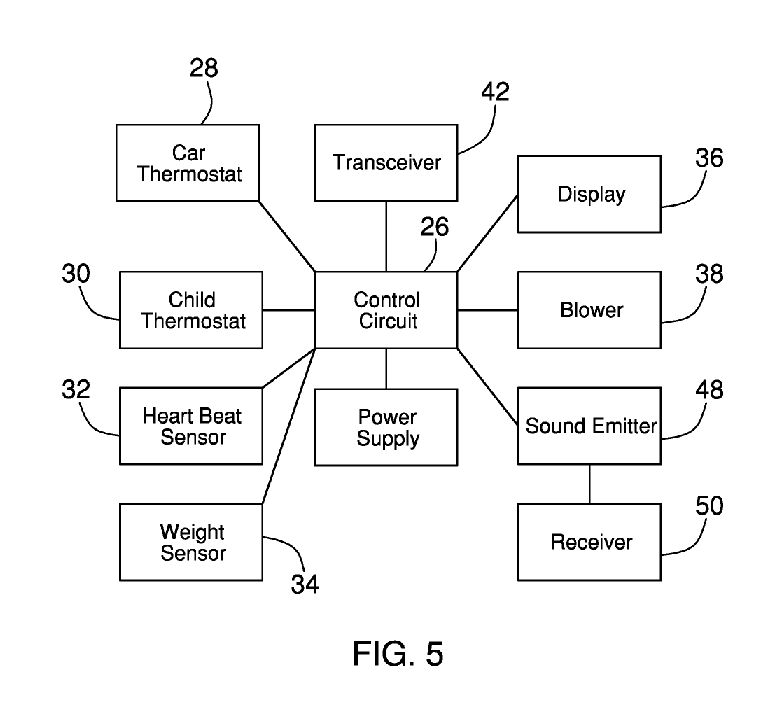

With reference now to the drawings, and in particular to FIGS. 1 through 5 thereof, a new car seat device embodying the principles and concepts of an embodiment of the disclosure and generally designated by the reference numeral 10 will be described.

As best illustrated in FIGS. 1 through 5, the health monitoring car seat assembly 10 generally comprises a child safety seat 12 that has an upper surface 14 and a lower surface 16 wherein the upper surface 14 receives a child thereon. A housing 18 is attached to the child safety seat 12 and has a base wall 20 and a peripheral wall 22 that extends outwardly from the base wall 20. The peripheral wall 22 is attached to the lower surface 16 of the child safety seat 12.

A sensing unit 24 is mounted on the housing 18. The sensing unit 24 detects when a mass is positioned in the child safety seat 12 and further detects vital signs of the mass in the child safety seat 12 when the mass is a child. The vital signs include body temperature and heart rate. Furthermore, the sensing unit 24 detects an ambient temperature of air adjacent to the child safety seat 12.

The sensing unit 24 comprises a control circuit 26 and a car thermostat 28 that is electrically coupled to the control circuit 26. The car thermostat 28 is in thermal communication with ambient air outside of the housing 18 and measures the temperature of the ambient air. A child thermostat 30 is electrically coupled to the control circuit 26 and is in thermal communication with a body of the child when the child is positioned in the child safety seat 12. The child thermostat 30 measures the body temperature of the child's body. A heartbeat sensor 32 is electrically coupled to the control circuit 26 and abuts the child when the child is in the child safety seat 12 to measure a heartbeat of the child. A weight sensor 34 is electrically coupled to the control circuit 26 and the weight sensor 34 detects when a mass of greater than 5.0 pounds is positioned in the child safety seat 12.

A display 36 is mounted on the child safety seat 12. The display 36 is electrically coupled to the sensing unit 24 and displays each of the ambient temperature, the temperature of the child, the heart rate of the child and the weight of the child. A blower 38 is mounted on the child safety seat 12 and blows air toward a central area 40 of the child safety seat 12 when turned on. The blower 38 is electrically coupled to the sensing unit 24 and turned on when the sensing unit 24 detects a dangerous ambient temperature. The dangerous ambient temperature is an ambient temperature greater than 90° F.

A transceiver 42 is mounted on the housing 18 and is detectable by an external navigation system. The external navigation system comprises a global positioning system wherein in an emergency situation the child safety seat 12 can be located using the global positioning system.

An extrinsic electronic device 44 is positioned remote of the housing 18 and has an audio file stored thereon. A sound emitting unit 46 is mounted on the child safety seat 2 and is in electrical communication with the extrinsic electronic device 44. The sound emitting unit 44 receives the audio file from the extrinsic electronic device 44 and emits the audio file when received.

The sound emitting unit 46 comprises a sound emitter 48 that is electrically coupled to the control circuit 26. The sound emitter 48 is mounted on the child safety seat 12. A receiver 50 is electrically coupled to the control circuit 26 and is electrically coupleable to the extrinsic electronic device 44. Moreover, the extrinsic electronic device 44 selectively sends the audio file to the receiver 50 wherein the sound emitter 48 emits the audio file.

A power supply 52 is mounted in the housing 18. The power supply 52 is electrically coupled to the sensing unit 24, the display 36, the blower, the transceiver 42 and the sound emitting unit 46. The power supply 52 comprises a battery wherein the battery is rechargeable.

In use, the child safety seat 12 is positioned in an interior of a vehicle. The child is placed on the upper surface 14 and is detected by the sensing unit 24. The sensing unit 24 detects each of the ambient temperature, the temperature of the child, the heart rate of the child and the weight of the child wherein the detected value is visible on the display 36. If the sensing unit 24 detects an ambient temperature over the dangerous ambient temperature the blower 38 is turned on to blow air toward the child to cool the child. The transceiver 42 can be detected by global positioning system to detect the child safety seat 12. Further, the sound emitting unit 48 is equipped to receive and play the audio file from the extrinsic electronic device 44.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. A child health monitoring car seat assembly comprising:
a child safety seat having an upper surface and a lower surface, said upper surface being configured to receive a child thereon;
a housing being attached to said child safety seat;
a sensing unit being mounted on said housing, said sensing unit being configured to detect when a mass is positioned in said child safety seat and further configured to detect vital signs of the mass in the child safety seat when the mass is a child, said vital signs including body temperature and heart rate, said sensing unit detecting an ambient temperature of air adjacent to said child safety seat;
a display being mounted on said child safety seat, said display being positioned adjacent to a top edge of said child safety seat wherein said display is configured to be viewable in a rear view mirror of a vehicle when said child safety seat is positioned in a rear seat of the vehicle, said display being electrically coupled to said sensing unit, said display displaying each of said ambient temperature, said temperature of the child, said heart rate of said child and said weight of said child;
a power supply being mounted in said housing, said power supply being electrically coupled to said sensing unit and said display.

2. The child health monitoring car seat according to claim 1, wherein said housing has a base wall and a peripheral wall extending outwardly from said base wall, said peripheral wall being attached to said lower surface of said child safety seat.

3. The child health monitoring car seat according to claim 1, wherein said sensing unit comprises:
a control circuit; and
a car thermostat being electrically coupled to said control circuit, said car thermostat being in thermal communication with ambient air outside of said housing and measuring said temperature of the ambient air.

4. The child health monitoring car seat according to claim 3, wherein sensing unit further includes a child thermostat being electrically coupled to said control circuit, said child thermostat being in thermal communication with a body of the child when the child is positioned in said child safety seat, said child thermostat measuring said body temperature of the child's body.

5. The child health monitoring car seat according to claim 4, wherein sensing unit further includes a heartbeat sensor being electrically coupled to said control circuit and abutting the child when the child is in said child safety seat to measure a heartbeat of the child.

6. The child health monitoring car seat according to claim 5, wherein sensing unit further includes a weight sensor being electrically coupled to said control circuit, said weight sensor detecting when a mass of greater than 5.0 pounds is positioned in said child safety seat.

7. The child health monitoring car seat according to claim 6, further including a blower being mounted on said child safety seat and blowing air toward a central area of said child safety seat when turned on, said blower being electrically coupled to said sensing unit.

8. The child health monitoring car seat according to claim 7, wherein said blower is turned on when said sensing unit detects a dangerous ambient temperature.

9. The child health monitoring car seat according to claim 8, wherein said dangerous ambient temperature is an ambient temperature greater than 90° F.

10. The child health monitoring car seat according to claim 1, further including a transceiver being mounted on said housing, said transceiver being configured to be detectable by an external navigation system, said external navigation system comprising a global positioning system.

11. The child health monitoring car seat according to claim 3, further including an extrinsic electronic device being positioned remote of said housing, said extrinsic electronic device having an audio file stored thereon.

12. The child health monitoring car seat according to claim 11, further including a sound emitting unit being mounted on said child safety seat, said sound emitting unit being in electrical communication with said extrinsic electronic device, said sound emitting unit receiving said audio file from said extrinsic electronic device and emitting said audio file when received.

13. The child health monitoring car seat according to claim 12, wherein said sound emitting unit comprises a sound emitter being electrically coupled to said control circuit, said sound emitter being mounted on said child safety seat.

14. The child health monitoring car seat according to claim 13, wherein said sound emitting unit further includes a receiver being electrically coupled to said control circuit and being electrically coupleable to said extrinsic electronic device, said extrinsic electronic device selectively sending said audio file to said receiver wherein said sound emitter emits said audio file.

15. (canceled)

16. The child health monitoring car seat according to claim 1, wherein said power supply comprises a battery, said battery being rechargeable.

17. A child health monitoring car seat assembly comprising:
a child safety seat having an upper surface and a lower surface, said upper surface being configured to receive a child thereon;
a housing being attached to said child safety seat, said housing having a base wall and a peripheral wall extending outwardly from said base wall, said peripheral wall being attached to said lower surface of said child safety seat;
a sensing unit being mounted on said housing, said sensing unit being configured to detect when a mass is positioned in said child safety seat and further configured to detect vital signs of the mass in the child safety seat when the mass is a child, said vital signs including body temperature and heart rate, said sensing unit detecting an ambient temperature of air adjacent to said child safety seat, said sensing unit comprising:
a control circuit;
a car thermostat being electrically coupled to said control circuit, said car thermostat being in thermal communication with ambient air outside of said housing and measuring said temperature of the ambient air;
a child thermostat being electrically coupled to said control circuit, said child thermostat being in thermal communication with a body of the child when the child is positioned in said child safety seat, said child thermostat measuring said body temperature of the child's body;
a heartbeat sensor being electrically coupled to said control circuit and abutting the child when the child is in said child safety seat to measure a heartbeat of the child;
a weight sensor being electrically coupled to said control circuit, said weight sensor detecting when a mass of greater than 5.0 pounds is positioned in said child safety seat;
a display being mounted on said child safety seat, said display being electrically coupled to said sensing unit, said display displaying each of said ambient temperature, said temperature of the child, said heart rate of said child and said weight of said child, said display being positioned adjacent to a top edge of said child safety seat wherein said display is configured to be viewable in a rear view mirror of a vehicle when said child safety seat is positioned in a rear seat of the vehicle;
a blower being mounted on said child safety seat and blowing air toward a central area of said child safety seat when turned on, said blower being electrically coupled to said sensing unit, said blower being turned on when said sensing unit detects a dangerous ambient temperature, said dangerous ambient temperature being an ambient temperature greater than 90° F.;
a transceiver being mounted on said housing, said transceiver being configured to be detectable by an external navigation system, said external navigation system comprising a global positioning system;
an extrinsic electronic device being positioned remote of said housing, said extrinsic electronic device having an audio file stored thereon;
a sound emitting unit being mounted on said child safety seat, said sound emitting unit being in electrical communication with said extrinsic electronic device, said sound emitting unit receiving said audio file from said extrinsic electronic device and emitting said audio file when received, said sound emitting unit comprising:
a sound emitter being electrically coupled to said control circuit, said sound emitter being mounted on said child safety seat;
a receiver being electrically coupled to said control circuit and being electrically coupleable to said extrinsic electronic device, said extrinsic electronic device selectively sending said audio file to said receiver wherein said sound emitter emits said audio file; and
a power supply being mounted in said housing, said power supply being electrically coupled to said sensing unit, said display, said blower, said transceiver and said sound emitting unit, said power supply comprising a battery, said battery being rechargeable.
